(19) United States
(12) Patent Application Publication
(10) Pub. No.: US 2024/0316901 A1
(43) Pub. Date: Sep. 26, 2024

(54) TIE LAYER COMPOSITIONS AND MULTILAYER TUBES INCORPORATING THE SAME

(71) Applicant: Nordson Corporation, Westlake, OH (US)

(72) Inventors: Thomas Bash, Huntington Beach, CA (US); Andrew Schombs, Placentia, CA (US); Lilian Campbell, Rancho Santa Margarita, CA (US)

(73) Assignee: Nordson Corporation, Westlake, OH (US)

(21) Appl. No.: 18/693,012

(22) PCT Filed: Oct. 3, 2022
(86) PCT No.: PCT/US2022/045499

§ 371 Date: Mar. 18, 2024

Related U.S. Application Data
(60) Provisional application No. 63/251,862, filed on Oct. 4, 2021.

(51) Int. Cl. B32B 7/12; A61L 29/04; A61L 29/08; B32B 1/08; B32B 15/02; B32B 15/08; B32B 27/08; B32B 27/28; B32B 27/32; B32B 27/34; B32B 27/40; B32B 37/12

(52) U.S. Cl. CPC B32B 7/12; A61L 29/041; A61L 29/085; B32B 1/08; B32B 15/02; B32B 15/08; B32B 27/08; B32B 27/285; B32B 27/322; B32B 27/34; B32B 27/40; B32B 37/1284; B32B 2255/10; B32B 2255/26; B32B 2274/00; B32B 2307/7376; B32B 2307/748; B32B 2325/00; B32B 2327/18; B32B 2535/00; B32B 2597/00

(57) ABSTRACT

Multilayer tubes including an inner layer and a tie layer are described. The inner layer includes a fluorinated polymer, and has an inner surface defining an inner diameter of the multilayer tube and an outer surface separated from the inner surface by a thickness of the inner layer. The tie layer includes a functionalized styrene block copolymer and is disposed on the outer surface of the inner layer. When the multilayer tube further includes an outer layer, the multilayer tubes exhibit improved peel strength as compared to multilayer tubes including the inner layer and outer layer alone. Methods of manufacturing the multilayer tubes and articles comprising the multilayer tubes are also described.

### BACKGROUND

Polymeric tubes are used in a number of applications, such as in intravascular medical devices. Various intravascular medical devices, including guide wires, catheters, and medical tubing, allow medical professionals to perform procedures such as delivery of a stent or other implantable device. In some cases, the implantable device is inserted into a patient's vasculature at a convenient site and delivered to a target site through the vasculature. A coating or liner on the inner wall of the catheter or medical tubing is commonly used to provide a smooth inner surface. The smooth inner surface may also reduce friction against the implantable device or other delivery components as they are pushed through the lumen of the catheter or medical tubing. One material that has been widely employed as a coating, tubing, or liner is polytetrafluorethylene (PTFE).

PTFE has a number of beneficial properties, including chemical resistance, high temperature resistance, biocompatibility, and a low coefficient of friction. In medical applications, the surface of PTFE tubing may be modified through an etching process to chemically activate the surface to increase adhesion strength to the outer jacket layers or other adjacent layers. However, the etched efficiency of the PTFE liner can be affected by ultraviolet (UV) light, moisture, and shelf life. Although it is recommended to apply adjacent layers as soon as possible after production of the PTFE liner to avoid degradation of efficiency, lead time in supply chains and other factors can make this difficult. As a result of the changed coefficient of friction, the adhesion between PTFE liners and the tubing or jackets in which they are employed may be less than desirable.

Accordingly, there is a need for PTFE-based liners having improved adhesion to polymeric jackets.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

The present disclosure discloses multilayer tubes including an inner layer comprising a fluorinated polymer (e.g., polytetrafluoroethylene (PTFE)) and a tie layer comprising a functionalized styrene block copolymer that is disposed on an outer surface of the inner layer. Multilayer tubes including the fluorinated polymer layer, the tie layer, and an additional polymeric layer can exhibit improved peel strength as compared to a fluorinated polymer layer and the additional polymeric layer alone.

In one exemplary aspect, a multilayer tube comprises an inner layer comprising a fluorinated polymer, wherein the inner layer has an inner surface defining an inner diameter of the multilayer tube and an outer surface separated from the inner surface by a thickness of the inner layer and a tie layer comprising a functionalized styrene block copolymer disposed on the outer surface of the inner layer.

In another exemplary aspect, a multilayer tube comprises an inner layer comprising a polytetrafluoroethylene (PTFE), wherein the inner layer has an inner surface defining an inner diameter of the multilayer tube and an outer surface separated from the inner surface by a thickness of the inner layer and a tie layer comprising a functionalized styrene block copolymer disposed on the outer surface of the inner layer.

In another exemplary aspect, a method of manufacturing a multilayer tube comprises forming a tie layer from a tie layer coating composition comprising a functionalized styrene block copolymer to an outer surface of an inner layer comprising a fluorinated polymer.

In another exemplary aspect, an intravascular medical device comprises the multilayer tube of any other aspect provided herein.

In another exemplary aspect, a catheter comprises the multilayer tube of any other aspect provided herein.

In another exemplary aspect, a medical tube comprises the multilayer tube of any other aspect provided herein.

In aspects, the functionalized styrene block copolymer of any other aspect provided herein comprises a maleic anhydride grafted styrene block copolymer. In aspects, the functionalized styrene block copolymer of any other aspect provided herein comprises maleic anhydride grafted poly(styrene-ethylene/butylene-styrene) (SEBS-g-MA). In aspects, the functionalized styrene block copolymer of any other aspect provided herein comprises greater than 25 wt % styrene.

In aspects, the multilayer tube of any other aspect provided herein further comprises an outer layer disposed on the tie layer such that the tie layer is disposed between the inner layer and the outer layer. In aspects, the outer layer of any other aspect provided herein comprises polyurethane, polyamide, polyether, polyamide/polyether block copolymer, polyester, co-polyester, stainless steel, glass, or combinations thereof. In aspects, the outer layer of any other aspect provided herein comprises a polyamide/polyether block copolymer.

In aspects, the tie layer of any other aspect provided herein has a thickness of from about 2.5 μm to about 30 μm.

In aspects, the multilayer tube of any other aspect provided herein further comprises a reinforcement layer. In aspects, the reinforcement layer of any other aspect provided herein comprises a wire coil.

In aspects, the outer surface of the inner layer of any other aspect provided herein is etched.

In aspects, the multilayer tube of any other aspect provided herein exhibits an increase in peel strength of at least about 25% compared to an otherwise identical multilayer tube not including the tie layer. In aspects, the multilayer tube of any other aspect provided herein exhibits an increase in peel strength of from about 25% to about 250% compared to an otherwise identical multilayer tube not including the tie layer.

In aspects, a method of manufacturing the multilayer tube of any other aspect provided herein comprises etching the outer surface of the inner layer prior to forming the tie layer.

In aspects, a method of manufacturing the multilayer tube of any other aspect provided herein comprises dipping the inner layer in the tie layer coating composition. In aspects, a method of manufacturing the multilayer tube of any other aspect provided herein comprises forming a reinforcement layer formed from a wire coil along at least a portion of a length of the multilayer tube. In aspects, forming the reinforcement layer of any other aspect provided herein comprises coiling a wire about the tie layer. In aspects, a method of manufacturing the multilayer tube of any other aspect herein comprises forming an outer layer comprising polyurethane, polyamide, polyether, or combinations thereof on the tie layer, such that the tie layer is disposed between the outer layer and the inner layer.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of the certain examples and implementations will be made by reference to various aspects of the appended drawings. It is appreciated that these drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 is an axial cross-section of a multilayer tube according to one or more aspects shown and described herein;

FIG. 2 is a bar graph showing the peel strength (Y-axis; in Newtons) for various samples (X-axis);

FIG. 3 is a bar graph showing the peel strength (Y-axis; in Newtons) for comparative samples incorporating PTFE liner A and a reinforcement layer as described in the Examples;

FIG. 4 is a bar graph showing the peel strength (Y-axis; in Newtons) for samples without a reinforcement layer and including various PTFE liners, tie layers, and outer layers as described in the Examples; and

FIG. 5 is a bar graph showing the peel strength (Y-axis; in Newtons) for samples incorporating PTFE liner B, a reinforcement layer, and a PEBAX outer layer as described in the Examples.

### DETAILED DESCRIPTION

The following description refers to the accompanying drawings, which illustrate example implementations of the present disclosure. Other implementations having different structures and operation do not depart from the scope of the present disclosure.

The present disclosure is directed to tie layers including a functionalized styrene block copolymer, multilayer tubes including the tie layers and a fluorinated polymer inner layer, methods of manufacturing multilayer tubes including the tie layers, and articles comprising the multilayer tubes. Multilayer tubes including the fluorinated polymer layer and an additional layer may exhibit improved peel strength as compared to otherwise identical multilayer tubes including the additional polymeric layer alone.

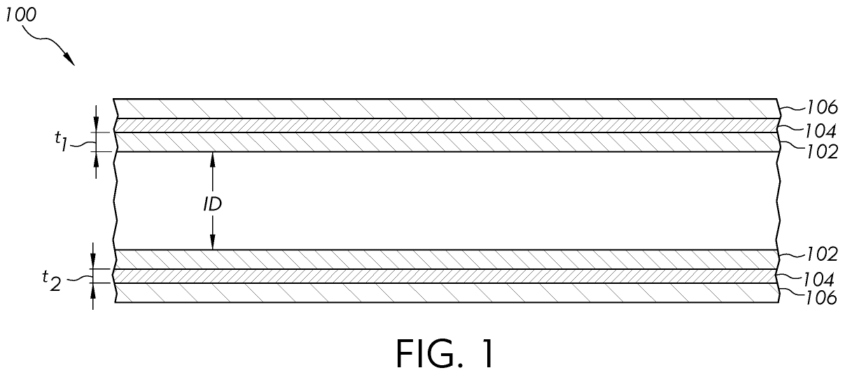

FIG. 1 depicts an axial cross-section of an exemplary multilayer tube 100. The multilayer tube 100 includes an inner layer 102, a tie layer 104, and an outer layer 106. The tie layer 104 is disposed between the inner layer 102 and the outer layer 106. In the present disclosure, the multilayer tube 100 may include the inner layer 102 and the tie layer 104. Accordingly, it should be understood that the outer layer 106 is optional in some aspects of the present disclosure. Moreover, other layers (e.g., a reinforcement layer, a fourth layer, fifth layer, or the like) may be added in accordance with the present disclosure.

The multilayer tube 100 extends axially along a length of the multilayer tube. The length of the multilayer tube 100 can vary widely and can be, for example 15 meters (m) or greater in length. The multilayer tube 100 also includes an inner diameter, ID, which is defined by an inner surface of the inner layer 102. Similarly, the inner diameter, ID, can vary in some aspects of the present disclosure. In some aspects of the present disclosure, the inner diameter, ID, is suitable for use in catheter applications. The multilayer tube 100 may have a generally cylindrical shape, although other shapes are contemplated and possible.

The inner layer 102 further includes an outer surface that is separated from the inner surface by a thickness, t₁, of the inner layer 102. The wall thickness can generally be described as being substantially uniform, and does not vary significantly around the circumference of the multilayer tube 100 or along the length of the multilayer tube 100.

In the present disclosure, the inner layer 102 generally comprises a fluorinated polymer, such as polytetrafluoroethylene (PTFE), perfluoroalkoxy alkane (PFA), polyvinylidene fluoride (PVDF), or fluorinated ethylene-propylene copolymer (FEP). The fluorinated polymer (sometimes referred to herein as a fluoropolymer) imparts a low coefficient of friction to the inner surface of the multilayer tube 100, while exhibiting high temperature and chemical resistance. Various fluorinated polymer resins are commercially available and can be used to form the fluorinated polymer tubes or liners provided herein. In various aspects of the present disclosure, the inner layer 102 consists essentially of fluorinated polymer, i.e., no additional components (e.g., fillers) are intentionally added to the inner layer 102.

As shown in FIG. 1, the tie layer 104 is disposed on an outer surface of the inner layer 102. In other words, the tie layer 104 is concentrically external to the inner layer 102 of the multilayer tube 100. In various aspects of the present disclosure, the tie layer 104 is disposed directly on an outer surface of the inner layer 102, with no intervening layers present between the tie layer 104 and the inner layer 102. The tie layer 104 has a thickness, t₂, that separates a first surface of the tie layer 104 from a second surface of the tie layer 104. In various aspects of the present disclosure, the thickness, t₂, of the tie layer 104 is from about 2.5 μm to about 30 μm, for example from about 2.5 μm to about 25 μm or from about 3 μm to about 20 μm, including any and all ranges and subranges therein.

The tie layer 104 is formed from a composition (sometimes referred to herein as a “tie layer coating composition”) that generally comprises a functionalized styrene block copolymer. In aspects of the present disclosure, the functionalized styrene block copolymer is a styrene block copolymer having a grafting compound attached thereto. Although a variety of grafting compounds may be known in the art, polar grafting compounds, and more particularly, maleated grafting compounds are used in various aspects of the present disclosure. In aspects of the present disclosure, the grafting compound is maleic anhydride. The amount of grafting compound in the functionalized styrene block copolymer can vary depending on the specific aspect, although in various aspects of the present disclosure, the grafting compound is present in an amount of greater than about 1 wt % and less than about 5 wt % based on a total weight of the functionalized styrene block copolymer, such as from about 1 wt % to about 3 wt %, including any and all ranges and subranges therein. In a particular aspect of the present disclosure, the functionalized styrene block copolymer includes from about 1.4 wt % to about 2 wt % of the grafting compound based on a total weight of the functionalized styrene block copolymer.

In the present disclosure, the styrene block copolymer may have a linear structure, although in some aspects, the styrene block copolymer may also encompass branched or radial polymers or functionalized block copolymers. In one or more aspects, the styrene block copolymer may comprise styrene-butadiene-styrene (SBS), styrene-isoprene-styrene (SIS), styrene-ethylene/butylene-styrene (SEBS), styrene-ethylene-propylene-styrene (SEPS), or combinations thereof. In aspects of the present disclosure, the styrene block copolymer is a styrene block copolymer having greater than or equal to 25 wt % styrene based on a total weight of the styrene block copolymer, such as greater than or equal to 30 wt % styrene. In some aspects of the present disclosure, the styrene block copolymer includes from 25 wt % to 50 wt % styrene, or from 30 wt % to 45 wt % styrene, including any and all ranges and subranges therein.

In particular aspects of the present disclosure, the functionalized styrene block copolymer is maleic anhydride grafted SEBS (SEBS-g-MA). Various commercially available functionalized styrene block copolymers may be suitable for use in the tie layer, including but not limited to KRATON FG1901 G Polymer, a linear triblock copolymer based on styrene and ethylene/butylene with a polystyrene content of 30% functionalized with from 1.0 wt % to 2.0 wt % maleic anhydride (e.g., from 1.0 wt % to 2.0 wt %, from 1.4 wt % to 2.0 wt %, from 1.0 wt. % to 1.7 wt %, or from 1.4 wt % to 1.7 wt %), or KRATON FG1924 G Polymer, a linear triblock copolymer based on styrene and ethylene/butylene with a polystyrene content of 13% functionalized with from 0.7 wt. % to 1.3 wt. % maleic anhydride (e.g., from 0.7 wt. % to 1.3 wt. %, from 1.0 wt. % to 1.3 wt. %, from 0.7 wt. % to 1.2 wt. %, or from 1.0 wt. % to 1.2 wt. %) available from Kraton Corporation (Houston, TX).

In various aspects of the present disclosure, the composition that forms the tie layer 104 (i.e., the tie layer coating composition) consists essentially of functionalized styrene block copolymer, i.e., no additional components (e.g., fillers) are intentionally added to the composition that forms the tie layer 104. In one particular aspect of the present disclosure, the composition that forms tie layer 104 consists essentially of SEBS-g-MA. In various aspects of the present disclosure, the composition that forms the tie layer 104 consists of functionalized styrene block copolymer, i.e., no additional components (e.g., fillers) are present in the composition that forms the tie layer 104. In one particular aspect of the present disclosure, the composition that forms the tie layer 104 consists of SEBS-g-MA. In various aspects of the present disclosure, the composition that forms the tie layer 104 excludes a tackifier. In various aspects of the present disclosure, the composition that forms the tie layer 104 excludes non-SEBS tackifiers. In particular aspects, the composition that forms the tie layer 104 excludes non-SEBS-g-MA tackifiers or tackifiers in addition to SEBS or SEBS-g-MA. In various aspects of the present disclosure, the composition that forms the tie layer 104 excludes ethylene/alpha olefin copolymer. In aspects of the present disclosure, the composition that forms the tie layer 104 excludes tackifiers and ethylene/alpha olefin copolymers.

Without being bound by theory, it is believed that, in addition to improving adhesion between the inner layer and the outer layer, the tie layer may also provide protection to the inner layer against adverse effects of UV light and/or moisture, thereby enabling the inner layer to have a longer shelf life.

In FIG. 1, the multilayer tube 100 further includes an optional outer layer 106. The outer layer 106 may be referred to as a “jacket,” and can be added to the multilayer tube 100 in order to confer different physical properties to the multilayer tube 100. For example, the outer layer 106 may impart strength or stiffness to the multilayer tube 100. The outer layer 106 can have any suitable thickness, and may extend along all or a portion of the length of the multilayer tube 100 when included. In aspects of the present disclosure, the outer layer 106 is constructed of one or more segments of different lengths and hardness, which can be used to impart different properties to different longitudinal sections of the multilayer tube 100.

The outer layer 106 may comprise any material known and used in the art. In aspects of the present disclosure, the outer layer 106 comprises stainless steel, glass, a polymer, or combinations or derivatives thereof. Polymers included in the outer layer 106 may include, by way of example and not limitation, polyurethane, polyamide, polyether, low density polyethylene (LDPE), high density polyethylene (HDPE), polyethylene terephthalate (PET), polyamide/polyether block copolymer, polyester, co-polyester, or combinations or derivatives thereof, including but not limited to polyamide/polyether block copolymers. Commercially available materials suitable for use include, by way of example and not limitation, products available under the tradenames TECOFLEX (an aliphatic polyether-based thermoplastic polyurethane available from The Lubrizol Corporation (Wickliffe, Ohio)), PEBAX (a polyether block amide available from Arkema S.A. (France)), and VESTAMID (a polyamide 12 available from Evonik Industries AG (Germany)).

In aspects of the present disclosure, the outer layer 106 consists essentially of polyurethane, polyamide, polyether, or combinations thereof, i.e., no additional components (e.g., fillers) are intentionally added to the outer layer 106. However, in some aspects, the outer layer 106 includes one or more additives, including but not limited to a radiopaque filler or radiopaque nanoclay, as may be known and used in the art. Radiopaque fillers include, without limitation, barium sulfate, bismuth subcarbonate, bismuth trioxides, bismuth oxychloride, tungsten, tantalum, platinum, gold, and combinations thereof.

Although referred to herein as an “outer layer,” it is contemplated that one or more additional layers may be disposed on the outer surface of the outer layer 106. For example, in some aspects of the present disclosure, a heat shrink layer (not shown) is placed over the outer layer to impart a radially inward force on the multilayer tube 100 during formation of the multilayer tube 100. The heat shrink layer may be formed from a fluoropolymer or polyolefin material such as polytetrafluoroethylene (PTFE) or fluorinated ethylene-propylene copolymer (FEP). The heat shrink layer may be referred to as a shape retention structure, as it retains the overall shape of the multilayer tube 100 during additional processing, as will be described in greater detail below.

A reinforcement layer in the form of a hypotube, coil or other reinforcement structure formed from stainless steel, nitinol, or another material may optionally be incorporated into the multilayer tube 100. When included, the reinforcement layer can provide support and/or structure to the multilayer tube 100. The reinforcement layer is positioned between the tie layer and the outer layer in various aspects of the present disclosure, although the reinforcement layer can be disposed in other positions within the multilayer tube construction. For example, in aspects of the present disclosure, the hypotube, coil, or other reinforcement structure is embedded into the inner layer 102 or the tie layer 104. In aspects of the present disclosure, the multilayer tube 100 excludes polymeric textiles. As used herein, the term “polymeric textiles” refers to materials made from polymeric fibers, threads, or yarns that are interlaced by any one of a variety of methods, including but not limited to weaving, knitting, or the like.

Coils can have any suitable pitch and corresponding surface area coverage, as may be known and used in the art. In aspects of the present disclosure, the coil has a surface area coverage of from 25% to 75%, from 30% to 70%, from 40% to 60%, or from 45% to 55%, including any and all ranges and subranges included therein. In one particular aspect, the multilayer tube 100 includes a stainless steel coil as a reinforcement layer.

The present disclosure is also directed to articles comprising the multilayer tubes. Examples of such articles include intravascular medical devices, including but not limited to, catheters and medical tubes. In aspects of the present disclosure, an intravascular medical device comprises the multilayer tubes of this disclosure. The intravascular medical device comprises catheters and/or medical tubes.

To form the multilayer tube 100, any one of a variety of methods may be employed, including but not limited to film casting and ram extrusion. In some aspects of the present disclosure, the inner layer 102 may be formed by extruding the material over a metallic substrate (e.g., a wire). Following extrusion, the substrate coated with the inner layer may be sintered at a temperature of greater than about 345° C. to melt the particles of the inner layer 102 and form a substantially uniform inner layer 102. The product is then cooled, and the inner layer 102 may be removed from the substrate. Other methods of forming the inner layer 102 are known and used in the art. Moreover, it is contemplated that a commercially available tube consisting essentially of the inner layer 102 may be obtained and processed to form the multilayer tube 100. Such commercially available tubes include, by way of example and not limitation, those available from Zeus Industrial Products, Inc. (Orangeburg, South Carolina), Nordson Medical (Easton, Pennsylvania), Junkosha Inc. (Japan), TE Connectivity Corporation (Berwyn, Pennsylvania), Medibrane Ltd. (Israel), Creganna Unlimited Company (Ireland), and Duke Extrusion (Santa Cruz, California).

In aspects of the present disclosure, the inner layer 102 is etched on the outer surface, although in other aspects of the present disclosure, the inner layer 102 is not subjected to etching or other surface modification processes. The etching can include, for example, chemically etching the inner layer by exposing the inner layer 102 to an etchant, such as fluorine, for a time effective to etch the outer surface of the inner layer to a depth of a few hundred angstroms or more (e.g., about 2.5×10⁻⁵ mm).

A tie layer coating composition is applied to the outer surface of the inner layer using any suitable method. For example, the tie layer coating composition can be applied via dip coating, spray coating, painting, drip coating, extrusion coating, or another suitable coating method. In aspects, the tie layer coating composition comprises the functionalized styrene block copolymer in the form of a dispersion. For example, the functionalized styrene block copolymer may be dispersed in a solvent (e.g., toluene) to form the tie layer coating composition. After application to the outer surface of the inner layer 102, the tie layer coating composition is dried to form the tie layer 104. In other aspects of the present disclosure, the tie layer coating composition is a molten form of the functionalized styrene block copolymer. After application to the outer surface of the inner layer 102, the molten tie layer coating composition cools and solidifies to form the tie layer 104.

In aspects including a reinforcement layer, the reinforcement layer is disposed on at least a portion of the surface of the tie layer 104. In some aspects, such as when the reinforcement layer is a wire coil, the reinforcement layer can be embedded into the tie layer 104. For example, the wire can be coiled around the multilayer tube before or during the drying of the tie layer composition to form the tie layer 104. In other aspects of the present disclosure, the reinforcement layer can be disposed on an outer surface of the tie layer 104 (e.g., coiled around the multilayer tube following the formation of the tie layer 104). In still other aspects of the present disclosure, the reinforcement layer can be disposed on a portion of the surface of the inner layer prior to the application of the tie layer coating composition.

In aspects including an outer layer 106, the outer layer 106 is formed by placing the material making up the outer layer over the tie layer 104. For example, the outer layer 106 may be placed on the tie layer 104 by applying the outer layer as a coating to the tie layer 104 and/or the reinforcement layer. Any suitable form of coating the outer layer 106 may be used as appropriate to achieve the desired outer layer 106. In other examples, the polymeric material can be in the form of a tube into which the multilayer tube including the inner layer 102 and the tie layer 104 is inserted. However, other methods of forming the outer layer 106 on the tie layer 104 may be used, depending on the particular aspect of the present disclosure.

A heat seal layer is further placed over the multilayer tube 100 in various aspects of the present disclosure. The multilayer tube 100, including the inner layer 102, the tie layer 104, the outer layer 106, and the heat seal layer, is then laminated or otherwise melt processed in accordance with the present disclosure.

The multilayer tubes of the present disclosure exhibit greater peel strength as compared to otherwise identical multilayer tubes not including the tie layer comprising functionalized styrene block copolymer. In aspects of the present disclosure, the peel strength is improved by greater than about 25% greater than about 50%, greater than about 55%, greater than about 100%, greater than about 150%, or even greater than about 200%. In aspects of the present disclosure, the peel strength is improved by from about 25% to about 250%, from about 30% to about 225%, from about 50% to about 225%, from about 100% to about 225%, from about 50% to about 110%, or any range or subrange within these ranges. In aspects of the present disclosure, the peel strength is greater than 8.5 Newtons (N), for example, greater than about 9.0 N, greater than about 9.5 N, greater than about 10.0 N, greater than about 10.5 N, greater than about 11.0 N, greater than about 12.0 N, greater than about 13.0 N, greater than about 14.0 N, greater than about 14.5 N, greater than about 15.0 N, greater than about 15.5 N, greater than about 16.0 N, greater than about 16.5 N, or greater than about 17.0 N. In aspects of the present disclosure, the peel strength is from about 8.5 N to about 20.0 N, from about 9.0 N to about 19.5 N, from about 9.5 N to about 19.0 N, from about 9.5 N to about 18.5 N, from about 10.0 N to about 18.0 N, from about 14.0 N to about 18.0 N, or any range or subrange within these ranges.

### Examples

The following examples are meant to better illustrate various aspects of the present disclosure, but are not intended to limit the scope of the present disclosure.

The following materials were employed in the examples that follow:
PTFE Liner A is a 0.254″ inner diameter ram extruded PTFE etched liner having a wall thickness of 0.035″ supplied by Zeus Industrial Products, Inc.;
PTFE Liner B is a 0.254″ inner diameter ram extruded PTFE etched liner having a wall thickness of 0.035″ supplied by Nordson Medical;
TECOFLEX EG-80A is an aliphatic polyether-based thermoplastic polyurethane available from The Lubrizol Corporation (Wickliffe, Ohio);
PEBAX 55D is a polyether block amide available from Arkema S.A. (France);
VESTAMID ML21 is a polyamide 12 available from Evonik Industries AG (Germany);
ESTANE 58810 is a thermoplastic polyurethane available from The Lubrizol Corporation (Wickliffe, Ohio); and
KRATON FG1901 G Polymer is a linear triblock copolymer based on styrene and ethylene/butylene with a polystyrene content of 30% functionalized with from 1.4 wt % to 2.0 wt % maleic anhydride, available from Kraton Corporation (Houston, TX).

Comparative samples (Samples C1-C12) using PTFE Liner A were obtained from Zeus including no tie layer, including a tie layer formed from TECOFLEX EG-80A, including a tie layer formed from PEBAX 55D, or including a tie layer formed from polyamide 12, as indicated in Table 1.

Additional comparative samples (Samples C13-C20) were prepared using PTFE Liner B alone (Samples C13-C14) or by applying a tie layer composition including ESTANE 58810 to PTFE Liner B (Samples C15-C20) while monitoring the viscosity. The inner diameter of the PTFE liner was unsupported and the ends were plugged to prevent the tie layer composition from contacting the inner surface of the PTFE liner. The tie layer had an average thickness of approximately 6 μm.

Six samples including the tie layer according to various aspects of the present disclosure (Samples 11-16) were prepared by applying a tie layer composition including KRATON FG1901 G Polymer to PTFE Liner B. In particular, the KRATON FG1901 G Polymer was solvated in a graduated cylinder and applied to the PTFE liner at a controlled extraction speed while the viscosity was monitored. The inner diameter of the PTFE liner was unsupported and the ends were plugged to prevent the tie layer composition from contacting the inner surface of the PTFE liner. The tie layer had an average thickness of approximately 7.5 μm.

Samples with and without reinforcement were evaluated, as indicated in Table 1. For samples with a reinforcement layer, a 0.004″×0.012″×0.024″ pitch 304 stainless steel coil was used, which provided a 50% surface area coverage.

Outer layers were selected from one of three different materials: TECOFLEX EG-80A, PEBAX 55D, and VESTAMID ML21, as indicated in Table 1.

TABLE 1
Sample Construction

| | | | Rein- | |
|---|---|---|---|---|
| | PTFE | | force- | |
| Sample | Liner | Tie Layer | ment | Outer Layer |
| C1 | A | None | None | TECOFLEX EG-80A |
| C2 | A | None | Yes | TECOFLEX EG-80A |
| C3 | A | None | None | PEBAX 55D |
| C4 | A | None | Yes | PEBAX 55D |
| C5 | A | None | None | VESTAMID ML21 |
| C6 | A | None | Yes | VESTAMID ML21 |
| C7 | A | TECOFLEX EG-80A | None | TECOFLEX EG-80A |
| C8 | A | TECOFLEX EG-80A | Yes | TECOFLEX EG-80A |
| C9 | A | PEBAX 55D | None | PEBAX 55D |
| C10 | A | PEBAX 55D | Yes | PEBAX 55D |
| C11 | A | VESTAMID ML21 | None | VESTAMID ML21 |
| C12 | A | VESTAMID ML21 | Yes | VESTAMID ML21 |
| C13 | B | None | None | PEBAX 55D |
| C14 | B | None | Yes | PEBAX 55D |
| C15 | B | ESTANE 58810 | None | TECOFLEX EG-80A |
| C16 | B | ESTANE 58810 | Yes | TECOFLEX EG-80A |
| C17 | B | ESTANE 58810 | None | PEBAX 55D |
| C18 | B | ESTANE 58810 | Yes | PEBAX 55D |
| C19 | B | ESTANE 58810 | None | VESTAMID ML21 |
| C20 | B | ESTANE 58810 | Yes | VESTAMID ML21 |
| I1 | B | KRATON FG1901 | None | TECOFLEX EG-80A |
| I2 | B | KRATON FG1901 | Yes | TECOFLEX EG-80A |
| I3 | B | KRATON FG1901 | None | PEBAX 55D |
| I4 | B | KRATON FG1901 | Yes | PEBAX 55D |
| I5 | B | KRATON FG1901 | None | VESTAMID ML21 |
| I6 | B | KRATON FG1901 | Yes | VESTAMID ML21 |

To construct the samples, the PTFE liner (with or without the tie layer, as indicated in Table 1), was stretched over a 0.63 mm (0.0249″) OD×30 cm (12″) length hypotube. The PTFE liner ranged in length from about 20 cm to about 28 cm (about 8″ to about 11″).

For samples including a reinforcement layer, the medial 12.7 cm (5″) of the liner was coiled with a 0.1 mm (0.004″)×0.3 mm (0.012″) 304 stainless steel wire with a 0.6 mm (0.024″) pitch. Coil ends were terminated with a laser weld operated at 180 V for 1.0 ms with a spot size of 0.30 mm in single pulse mode.

The outer layer indicated for each sample in Table 1, having a length similar to that of the corresponding PTFE liner, was placed over the sample. A 17.7 cm (7″) long piece of 9.5 mm (⅜″) FEP heat shrink was then placed over the sample. The medial 12.7 cm (5″) of each sample was laminated using a vertical laminator. Lamination parameters were determined based on the material of the outer layer, as indicated in Table 2. Samples were removed from the laminator, the heat shrink layer was removed, and the sample was cut in half for testing.

TABLE 2
Lamination Parameters

| Outer Layer | Temperature (° C.) | Speed (mm/s) |
|---|---|---|
| TECOFLEX EG-80A | 237.8 | 0.8 |
| PEBAX 55D | 248.9 | 0.8 |
| VESTAMID ML21 | 260 | 1 |

The peel strength of 5 units per test was measured using the Instron Tensile test using the parameters set forth in Table 3.

TABLE 3
Peel Strength Test Parameters

| Parameter | Value |
|---|---|
| Gauge Length | 50.8 mm (2″) |
| Crosshead speed | 250 mm/min |
| Grip pressure | 6.89 kPa (60 psi) |
| End condition | 100% reduction in rate of force |
| Sampling Rate Criteria 1 | Time: 100 ms |
| Sampling Rate Criteria 2 | Force: 0.13N |

To prepare the samples including the reinforcement layer for testing, the liner was peeled apart from the outer layer until the first few coil layers were exposed in order to ensure that the samples included only data from delamination of the coiled sections. Displacement and force values were zeroed. Samples were then clamped into the pneumatic grips with the bottom grip clamping the PTFE liner and the top grip clamping the outer layer of the corresponding sample. The samples were ensured to have no slack within the PTFE liner or the outer layer.

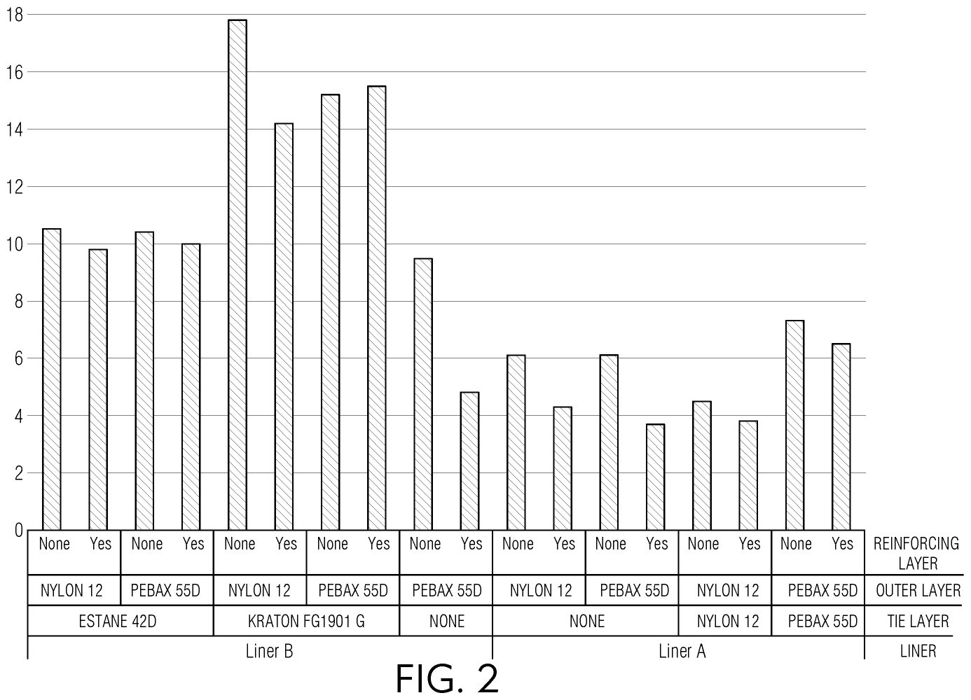

The resultant data is presented in Table 4 and FIGS. 2-5. In particular, the values reported in Table 4 are the average values of a specific 20 mm range of displacement. Due to the variability of the data, the displacement range varied from test to test, but is defined as the first 20 mm increment that occurred 10 mm after all test samples in the same data set have stabilized. This average value is reported, as it provided a consistent range of values. The values reported in Table 4 are consistent with the values obtained by averaging force values with the initial ramp up and ramp down values excluded by including only values above a specified force value in the averaging, except for the values for the TECOFLEX EG-80A outer layer. Regarding the data obtained for the samples including the TECOFLEX EG-80A outer layer, it is believed that the elasticity of the outer layer causes noise and oscillations in the data and as a result, the data was excluded.

TABLE 4
Peel Strength Test Results

| | |
|---|---|
| Without Reinforcement Layer | With Reinforcement Layer |

| | Average Peel Force | | Average Peel Force |
|---|---|---|---|
| Sample | (N) | Sample | (N) |
| C1 | 12.0 | C2 | 22.9 |
| C3 | 6.1 | C4 | 3.7 |
| C5 | 6.1 | C6 | 4.3 |
| C7 | 14.5 | C8 | 22.6 |
| C9 | 7.3 | C10 | 6.5 |
| C11 | 4.5 | C12 | 3.8 |
| C13 | 9.5 | C14 | 4.8 |
| C15 | 5.0 | C16 | 3.8 |
| C17 | 10.4 | C18 | 10.0 |
| C19 | 10.5 | C20 | 9.8 |
| I1 | n/a | I2 | 17.2 |
| I3 | 15.2 | I4 | 15.5 |
| I5 | 17.8 | I6 | 14.2 |

As illustrated in Table 4, the addition of the reinforcement layer (e.g., a coil wire) was found to reduce peel strength for outer layers of PEBAX 55D, and VESTAMID ML21. Without being bound by theory, it is believed that the reinforcement layer reduces the ability of the PTFE liner to bond to the outer layer. In particular, visual inspection of the samples showed that sections where the liner is in contact with the reinforcement layer, the etch in the liner remained, while the etch was removed during peel testing in sections where the outer layer was in contact with the liner.

However, as shown in FIG. 2 and Table 5, adding a tie layer greatly reduced the negative effect of the reinforcement layer on the peel strength. In particular, samples including a tie layer (including tie layers formed from TECOFLEX EG-80A, PEBAX 55D, VESTAMID ML21, ESTANE 58810, and KRATON FG1901) exhibited an average reduction of peel strength of only 9.21% as a result of the addition of the reinforcement layer as compared to the 39.44% average reduction of peel strength exhibited by samples not including a tie layer but including the reinforcement layer.

TABLE 5
Comparison of Impact of Various Tie Layer Chemistries

| | | | Average for |
|---|---|---|---|
| | | Increase | Reinforced |
| | Reinforcement | of Peel | and Non- |
| Tie Layer | Layer | Strength | Reinforced |
| PEBAX 55D | No | 19.67% | 47.67% |
| | Yes | 75.68% | |
| VESTAMID ML21 | No | −26.23% | −18.93% |
| | Yes | −11.68% | |
| ESTANE 58810 | No | 9.47% | 58.90% |
| | Yes | 108.33% | |
| KRATON FG1901 | No | 60.00% | 141.16% |
| | Yes | 222.92% | |

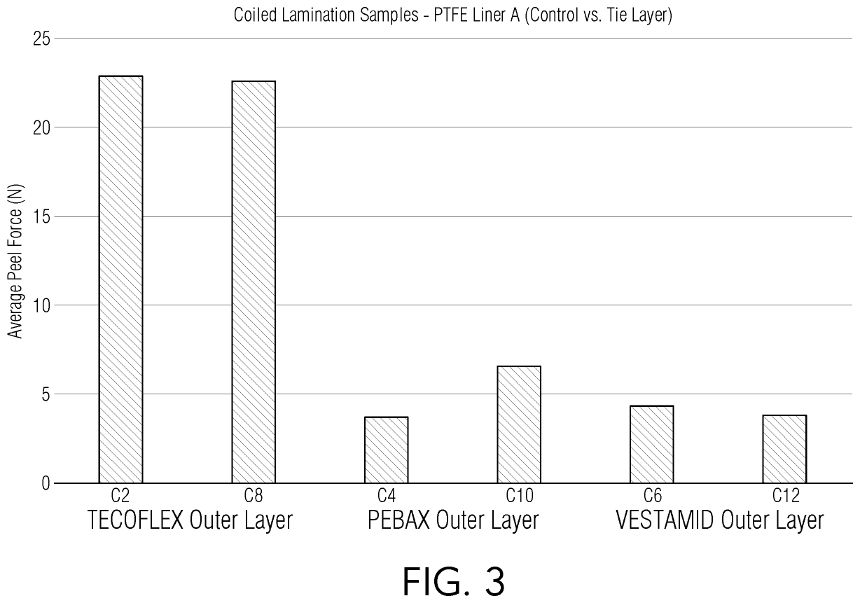

However, the tie layer increased the adhesion by varying amounts, as shown in FIG. 2-5 and Table 5. In particular, the tie layer including KRATON FG1901 improved the peel strength of the liners as compared to liners having tie layers including TECOFLEX EG-80A, PEBAX 55D, VESTAMID ML21, or ESTANE 58810. Notably, the inclusion of the VESTAMID ML21 as a tie layer (samples C11 and C12) actually reduced the peel strength as compared to an otherwise identical multilayer tube not including the VESTAMID ML21 tie layer (e.g., samples C5 and C6). The effect of the use of tie layers including TECOFLEX EG-80A, PEBAX 55D, VESTAMID ML21 on PTFE liner A including a reinforcement layer and an outer layer that corresponds to the tie layer is shown in FIG. 3.

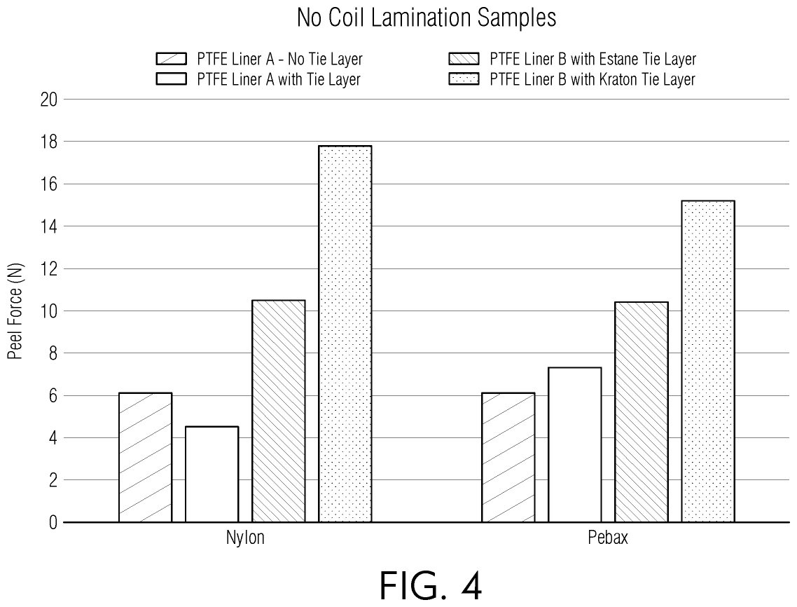

FIG. 4 shows the peel strength of various samples without a reinforcement layer with a VESTAMID (nylon) and PEBAX outer layer. As shown in FIG. 4, the samples including a KRATON tie layer exhibit increased peel strength for both outer layer chemistries.

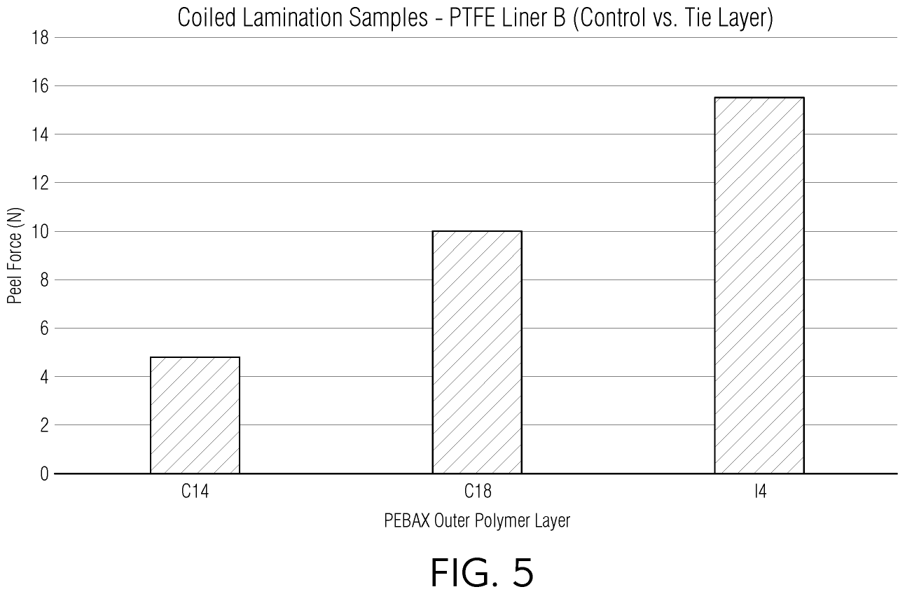

FIG. 5 shows the peel strength of various samples including PTFE liner B, a reinforcement layer, and a PEBAX outer layer. Although the ESTANE tie layer (C18) exhibits an increased peel strength over the control (C14), the use of the KRATON tie layer (I4) results in an increased peel strength over even the ESTANE tie layer.

It was noted that the baseline adhesion for the samples including PTFE liner B was greater than the baseline adhesion for the samples including PTFE liner A, for samples both including and excluding a reinforcement layer (55.74% and 29.73%, respectively).

While various inventive aspects, concepts and features of the disclosures may be described and illustrated herein as embodied in combination in the example aspects of the present disclosure, these various aspects, concepts, and features may be used in many alternative aspects of the present disclosure, either individually or in various combinations and sub-combinations thereof. Unless expressly excluded herein all such combinations and sub-combinations are intended to be within the scope of the present application. Still further, while various alternative aspects of the present disclosure as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative aspects of the present disclosure, whether presently known or later developed. Those skilled in the art may readily adopt one or more of the inventive aspects, concepts, or features into additional aspects of the present disclosure and uses within the scope of the present application even if such aspects of the present disclosure are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description in the specification.

1. A multilayer tube comprising:
an inner layer comprising polytetrafluoroethylene (PTFE), wherein the inner layer has an inner surface defining an inner diameter of the multilayer tube and an outer surface separated from the inner surface by a thickness of the inner layer; and
a tie layer comprising a functionalized styrene block copolymer disposed on the outer surface of the inner layer.

2. The multilayer tube according to claim 1, wherein the functionalized styrene block copolymer comprises a maleic anhydride grafted styrene block copolymer.

3. The multilayer tube according to claim 2, wherein the functionalized styrene block copolymer comprises maleic anhydride grafted poly(styrene-ethylene/butylene-styrene) (SEBS-g-MA).

4. The multilayer tube according to claim 1, wherein the functionalized styrene block copolymer comprises greater than 25 wt % styrene.

5. The multilayer tube according to claim 1, further comprising:
an outer layer disposed on the tie layer such that the tie layer is disposed between the inner layer and the outer layer.

6. The multilayer tube according to claim 5, wherein the outer layer comprises polyurethane, polyamide, polyether, polyamide/polyether block copolymer, polyester, co-polyester, stainless steel, glass, or combinations thereof.

7. (canceled)

8. The multilayer tube according to claim 1, wherein the tie layer has a thickness of from about 2.5 μm to about 30 μm.

9. The multilayer tube according to claim 1, further comprising a reinforcement layer.

10. The multilayer tube according to claim 9, wherein the reinforcement layer comprises a wire coil.

11. The multilayer tube according to claim 1, wherein the outer surface of the inner layer is etched.

12. (canceled)

13. The multilayer tube according to claim 1, wherein the multilayer tube exhibits an increase in peel strength of from about 25% to about 250% compared to an otherwise identical multilayer tube not including the tie layer.

14. A method of manufacturing a multilayer tube comprising:
forming a tie layer from a tie layer coating composition comprising a functionalized styrene block copolymer to an outer surface of an inner layer comprising polytetrafluoroethylene (PTFE).

15. The method according to claim 14, further comprising:
etching the outer surface of the inner layer prior to forming the tie layer.

16. The method according to claim 14, wherein forming the tie layer comprises dipping the inner layer in the tie layer coating composition.

17. The method according to claim 14, further comprising:
forming a reinforcement layer formed from a wire coil along at least a portion of a length of the multilayer tube.

18. (canceled)

19. The method according to claim 14, further comprising:
forming an outer layer comprising polyurethane, polyamide, polyether, or combinations thereof on the tie layer, such that the tie layer is disposed between the outer layer and the inner layer.

20. (canceled)

21. An intravascular medical device comprising the multilayer tube of claim 1.

22. A catheter comprising the multilayer tube of claim 1.

23. A medical tube comprising the multilayer tube of claim 1.

24. A multilayer tube comprising:
an inner layer comprising a fluorinated polymer, wherein the inner layer has an inner surface defining an inner diameter of the multilayer tube and an outer surface separated from the inner surface by a thickness of the inner layer; and
a tie layer comprising a functionalized styrene block copolymer disposed on the outer surface of the inner layer.

25. (canceled)
